(19) United States
(12) Patent Application Publication
(10) Pub. No.: US 2020/0261276 A1
(43) Pub. Date: Aug. 20, 2020

(54) Post-Operative Hybrid Dressing To Optimize Skin-Grafting Procedures In Reconstructive Surgery

(71) Applicant: University of Massachusetts, Boston, MA (US)

(72) Inventors: Jorge Rigel Lujan Hernandez, Worcester, MA (US); Raymond M. Dunn, Worcester, MA (US)

(21) Appl. No.: 16/762,003

(22) PCT Filed: Nov. 8, 2018
(86) PCT No.: PCT/US2018/059812

§ 371 Date: May 6, 2020

Related U.S. Application Data
(60) Provisional application No. 62/583,376, filed on Nov. 8, 2017.

(51) Int. Cl. A61F 13/00; A61F 13/02; A61M 1/00

(52) U.S. Cl. CPC A61F 13/00068; A61F 13/0216; A61F 2013/00174; A61M 1/009; A61M 1/0072; A61M 1/0068

(57) ABSTRACT

A device and associated method for treating a skin graft donor site includes a sheet having dimensions to cover the skin graft donor site and an area of skin surrounding the skin graft donor site. An adhesive is provided that has properties of adhering to the skin and the sheet in a manner that forms a liquid impermeable boundary surrounding the skin graft donor site. A port is integrated with the sheet that enables a suction force to be applied therethrough to produce a negative pressure between the sheet and skin graft donor site to manage fluid produced by the skin graft donor site.

### RELATED APPLICATION

This application claims the benefit of U.S. Provisional Application No. 62/583,376, filed on Nov. 8, 2017. The entire teachings of the above application are incorporated herein by reference.

### BACKGROUND OF THE INVENTION

Every year, millions of victims suffer physically and emotionally as a consequence of trauma, burns, cancer or chronic wounds. Skin-grafting is the most commonly performed procedure to reconstruct body parts and restore functionality after these disfiguring events. This technique plays an important role by providing coverage with healthy skin to the affected area after an accident, or after a large cancer resection where uncomplicated wound healing on its own would be near impossible.

Although skin-grafting has associated complications, such as post-operative pain, infection, and requirement of additional grafting sessions, the main complication that is found almost in 100% of cases is the discomfort caused by the donor site from where the skin is taken. Most of the research on improvement of skin grafting techniques has been aimed toward the graft itself, and not toward the donor site. Healthy skin is harvested from the “donor site” by using a fine blade that transects the skin with precision to a specified depth, including parts of the two outermost layers of skin—the epidermis and dermis. Then, the donor site is left to heal by itself by means of re-epithelialization.

Clinical evidence has shown that the donor site constitutes the main source of discomfort after the procedure for several reasons: it is an acute wound that is highly exudative, requiring multiple dressing changes to keep the minimal necessary moisture; it is very sensitive and dressings changes or simple contact becomes extremely painful; it is prone to dangerous pathogen infections such as pseudomonas, Staphylococcus aureus, and fungi if moisture accumulates excessively.

Approaches for addressing the donor site have been divided into moist (non-absorbent) and dry dressings (absorbent to super absorbent). While absorbent dressings eliminate excess fluid, they eventually saturate and have to be exchanged more often, which induces more pain. On the other hand, non-absorbent dressings are can sooth the pain. Moreover, non-absorbent dressings do not absorb liquid, generating accumulation and constant leakage onto the patient and the patient's bed, requiring uncomfortable multiple nursing/physician interventions.

Significance: At one example institution, at least 400 skin grafts are performed yearly by not only plastic surgeons, but also orthopedic surgeons and general surgeons. The associated complications of the donor site result in higher narcotic requirement, lower mobility, prolonged rehabilitation process, increased nursing/physician interventions, uncomfortable dressing changes, risk of infection, and requirement of additional procedures, resource utilization, and overall unpleasant patient experience. Although skin grafts were first described in the 1800's, the strategies used in modern medicine for the management of donor sites have remained unchanged for decades. Yet, the multiple inconveniences occasioned by skin graft donor sites continue to persist.

### SUMMARY OF THE INVENTION

The invention is generally directed to a device and associated method for treating a skin graft donor site. The device, which in one embodiment can be a hydrogel dressing, is sized and configured for application to a skin graft donor site, which is a main source of complications with an unmet need.

An embodiment of the device includes a sheet having dimensions to cover the skin graft donor site and an area of skin surrounding the skin graft donor site. An adhesive is provided that has properties of adhering to the skin and the sheet in a manner that forms a liquid impermeable boundary surrounding the skin graft donor site. A port is integrated with the sheet that enables a suction force to be applied therethrough to produce a negative pressure between the sheet and skin graft donor site to manage fluid produced by the skin graft donor site.

The term “port” as used herein refers to an opening and can be in the form of a “port structure” that defines the opening. The term “port” and “port structure” are used interchangeably.

The port can be sized to pass up to 100 ml of fluid per day. In some embodiments, the suction applied through the port can produce a negative pressure of less than 100 mm Hg.

The port can include multiple ports. The multiple ports can have a higher density in the sheet toward the boundary than toward a center of the skin graft donor site.

The device can further include a structure to prevent clogging at the port.

The device can further include filaments at the sheet, which are configured in the form of channels for fluid to pass therethrough from the skin graft donor site to the port. The filaments can be embedded in the sheet.

The device can further include a reservoir in fluid communication with the port.

The device can include a bulb defining the reservoir and being configured to provide a negative pressure to apply the suction force through the port.

In some embodiments, the reservoir is integrated into the sheet and positionable apart from the skin graft donor site.

The device can further include a reservoir base sheet, configured to be fixedly coupled to skin apart from the skin surrounding the skin graft donor site, and a reservoir top sheet configured to be detachably coupled to the reservoir base sheet, the reservoir being defined between the reservoir base sheet and the reservoir top sheet in at least one location the reservoir top sheet is not coupled to the reservoir base sheet.

Any sheet of the device, such as the reservoir top sheet, can be formed from a vapor transmissible film.

The device can further include one or more filaments between the port and the reservoir.

The device can further include additional material, such as bio-resorbable material, configured to be positioned over the skin graft donor site and under the sheet.

The device can further include biomaterials with integrated growth factors to enhance epidermal healing coupled to the sheet.

The sheet can include a material selected from a group of materials consisting of: a polyurethane, an organic polymer, a hydrogel.

The adhesive can be on the sheet other than over a portion of the sheet expected to cover the skin graft donor site. The adhesive can be provided on a backing sheet and configured to release from the backing sheet following contact of the adhesive to the skin surrounding the skin graft donor site.

In any of the embodiments, the sheet can include transparent material to allow visualization of the donor site without removal of the sheet or the device.

A drug or molecule can be embedded in the sheet for delivery to the skin graft donor site.

The device can further include a removable drain device, e.g., a tube or other conduit, in fluid communication with the port to drain fluid from the skin graft donor site. When the skin draft donor site is not draining anymore, the drain can be detached from the port, without the need to exchange the remainder of the device. The device can include a mechanism, e.g., a valve or seal, to close the port when the drain is detached.

An embodiment of the method for treating the skin graft donor site includes adhesively coupling a sheet to an area of skin surrounding the skin graft donor site to form a liquid impermeable boundary, and applying a suction force through a port integrated with the sheet to produce a negative pressure between the sheet and skin graft donor site to manage fluid produced by the skin graft donor site.

The method can further include delivering a drug or a molecule to the skin graft donor site, wherein the drug or molecule is embedded in the sheet or delivered through the port.

In an embodiment, the device serves as a hydrogel dressing that includes a hydrogel sheet defining channels in fluid communication with an opening that is also defined by the hydrogel sheet.

Example advantages of some embodiments of the invention include: reduction of leakage of excess fluid; reduction of pain, reduction of requirement of dressing changes. These advantages translate into decreased nursing/house staff interventions, decreased opioid use, faster recovery, and faster re-epithelialization.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a schematic illustration of a patient having a wound to which a skin graft was applied from a skin graft donor site of the patient.

FIG. 1B is a schematic top view of a device for treating a skin graft donor site according to an example embodiment of the invention.

FIG. 1C is a sectional view of the device of FIG. 1B applied to the skin graft donor site.

FIG. 2A is a schematic top view of a device for treating a skin graft donor site illustrating filaments configured in the form of channels according to an example embodiment of the invention.

FIG. 2B is a sectional view of the device of FIG. 2A.

FIGS. 2C and 2D are sectional views illustrating example filaments configured in the form of channels positioned over a skin graft donor site.

FIG. 2E is a schematic top view of a device including filaments and a reservoir for treating a skin graft donor site according to an example embodiment of the invention.

FIG. 3 is a top view of a device for treating a skin graft donor site schematically illustrating multiple ports according to an example embodiment of the invention.

FIG. 4A is a top view of a device for treating a skin graft donor site schematically illustrating a bulb for providing a suction force through a port of the device, according to an example embodiment of the invention.

FIG. 4B illustrates the device of FIG. 4A used on a patient.

FIG. 5A schematically illustrates a top view of a device having a reservoir in fluid communication with a port for treating a skin graft donor site according to an example embodiment of the invention.

FIG. 5B is sectional view of the device of FIG. 5A illustrating the reservoir defined between a base sheet and a top sheet.

FIG. 6 is a schematic illustration of a device having an integrated reservoir for treating a skin graft donor site according to an example embodiment of the invention.

FIG. 7 is a schematic illustration of components of a device for treating a skin graft donor site including additional material positioned over the skin graft donor site and under the sheet of the device.

FIGS. 8A-8D schematically illustrate application of a device, which includes an adhesive provided on a backing sheet, to a skin graft donor site.

FIG. 9A is schematic illustration of a device for treating a skin graft donor site including a hydrogel positioned over the skin graft donor site.

FIG. 9B is a sectional view of the device of FIG. 9A.

FIGS. 10A and 10B schematically illustrate treating a skin graft donor site with a device that includes a hydrogel to which suction is applied.

FIG. 10C is a sectional view of the device of FIG. 10A.

### DETAILED DESCRIPTION OF THE INVENTION

A description of example embodiments follows.

The invention generally provides an enhanced wound treatment dressing for skin grafts, targeting the skin graft donor site. Described are embodiments of a dressing device based upon wound healing principles from the inventors' cumulative clinical experience after many years of patient care as well as the inventors' expertise with bioengineered materials. Preliminary evidence has shown that there is no ideal way of addressing the donor site. Moreover, there are several well-known principles that should be considered for ideal wound healing of skin grafts donor sites.

Donor site healing principles:

Principle 1: Moisture is needed. Wounds produce exudate that contains growth factors known to stimulate wound healing. Wounds also heal faster in a moist environment through promoting keratinocyte migration and re-epithelialization.

Principle 2: The donor site is an acute wound. It produces a high amount of fluid exudate and moderate bleeding, which, in accumulation, generates an infection-prone environment.

Principle 3: Current management involves non-absorbent or absorbent dressings. Non-absorbent dressings accumulate a high amount of exudate. This exudate soon leaks through the dressing site, causing high levels of patient discomfort and requiring constant dressing changes and nursing interventions.

Principle 4: If absorbent dressings are used, they become saturated quickly and require multiple changes. These dressing changes are very painful and require sterile technique and trained personnel.

Principle 5: If left open to air, the wound dries out, hampering re-epithelialization and being exposed to pathogens and shearing damage.

An ideal dressing should share characteristics of both dry and moist dressings. The dressing should not only address the exudate problem but expedite painless healing of the donor site by maintaining a moist and sterile environment. In some embodiments, a hybrid dressing is provided that combines features from both types of dressings. This novel concept reduces discomfort, nursing interventions, pain, frequency of dressing changes, and promote adequate epithelialization. This will overall reduce complications and improve patient experience.

Embodiments of the present invention address the problem of managing fluid, e.g., exudate, which is produced by a surface wound, such as a split thickness skin graft donor site. Other approaches have addressed drainage of a cavity wound, such as surgical wounds from abdominal surgery. For example, a surgical drain device that includes an adhesion matrix of biodegradable polymer material and drain tubes attached to the matrix is described in International Application Number PCT/US2012/033608, filed Apr. 13, 2012 and entitled “Surgical Cavity Draining And Closure System,” which published as WO 2012/142473 A1 on Oct. 18, 2012.

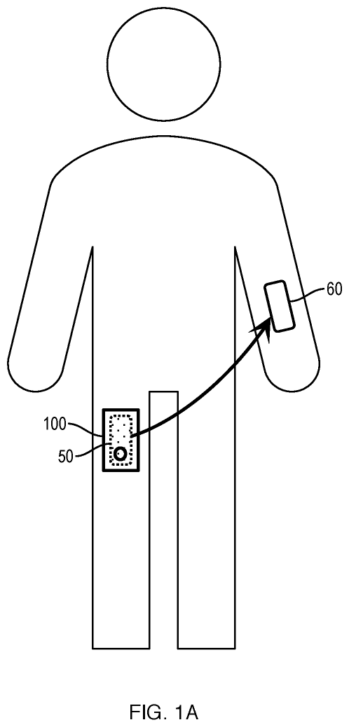

FIG. 1A illustrates a patient with two wound sites: a burn or other wound site 60 that required a skin graft dressing (i.e., a skin graft is already applied), and the skin graft donor site 50 from which the skin graft was taken (as illustrated by the arrow). The invention is generally directed to a device 100 for treating the skin graft donor site 50. The skin graft donor site tends to be a smooth wound that produces significant amounts of fluid over the first five or six days after the skin graft was taken. The device 100 solves the problem of fluid management associated with the skin graft donor site 50.

FIGS. 1B and 1C illustrate a device 100 for treating a surface wound, such as a skin graft donor site 50. A device 100 includes a sheet 102, an adhesive 104, and a port 106. The sheet 102 has dimensions to cover the skin graft donor site 50 and an area of skin 55 surrounding the skin graft donor site. The adhesive 104 has properties of adhering to the skin and the sheet 102 in a manner that forms a liquid impermeable boundary 108 surrounding the skin graft donor site 50. The port 106 is integrated with the sheet 102 and enables a suction force 110 to be applied through the port 106 to produce a negative pressure between the sheet 102 and skin graft donor site 50 to manage fluid produced by the skin graft donor site. In an embodiment, the port is sized to pass up to 100 ml of fluid per day. In an embodiment, the applied suction force is such that a negative pressure between the sheet and the donor site is less than 100 mm Hg.

As shown in FIGS. 1B and 1C, the adhesive 104 is on the sheet 102 other than over a portion of the sheet expected to cover the skin graft donor site so that the adhesive does not contact the sensitive skin graft donor site 50. At least a portion of material composing the sheet 102 is biocompatible, such that material that is in contact with the skin graft donor site 50 does not introduce a biocompatibility issue. The sheet 102 can include transparent material to allow visualization of the donor site without removal of the sheet 102 or other parts of the device.

For example, the sheet 102 can include a material selected from a group of materials consisting of: a polyurethane, an organic polymer, a hydrogel.

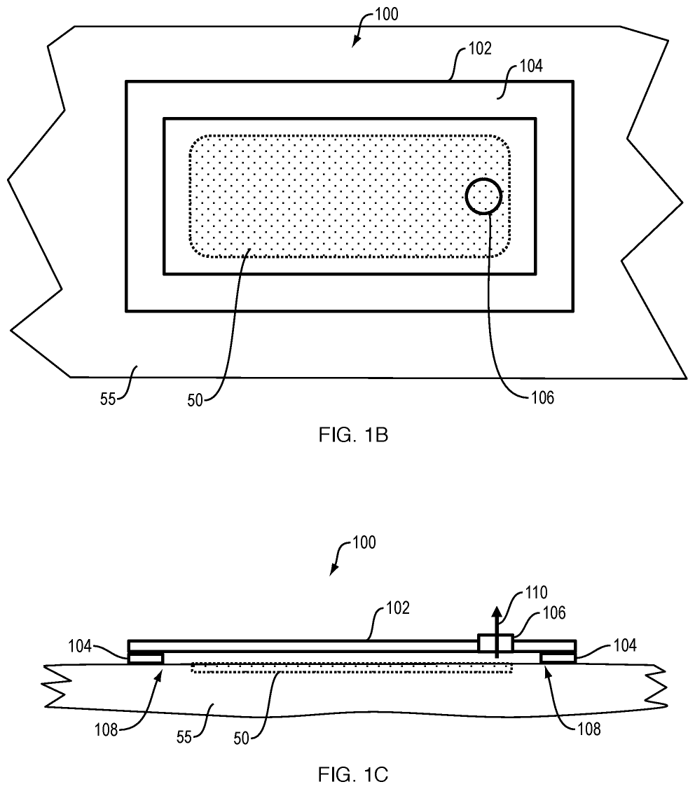

As illustrated in FIGS. 2A and 2B, a device 200 for treating a skin draft donor site can further include filaments 212 at the sheet 102. The filaments 212 are configured in the form of channels 214 for fluid to pass therethrough from the skin graft donor site 50 to the port 106. As in the device of FIGS. 1B and 1C, the sheet 102 of device 200 has dimensions to cover the skin graft donor site 50 and an area of skin 55 surrounding the skin graft donor site. The adhesive 104 has properties of adhering to the skin and the sheet 102 in a manner that forms a liquid impermeable boundary 108 surrounding the skin graft donor site 50.

FIGS. 2C and 2D are sectional views illustrating example filaments 212 configured in the form of channels 214. As shown, the filaments 212 are provided at the sheet 102 and are configured to be positioned over the skin graft donor site 50. The sheet 102 is flexible and drapes over the filaments 212, thereby providing channels 214 for fluid to pass from the donor site 50 to the port 106. Filaments having a circular cross-section are illustrated in FIG. 2C. Filaments having a triangular cross-section are illustrated in FIG. 2D. The filaments and channels can also be embedded in the sheet.

FIG. 2E is a schematic top view of a device 200′ including filaments 212 and a reservoir 216 for treating a skin graft donor site according to an example embodiment of the invention. The reservoir 216 is in fluid communication with the port 106 via a conduit 222, which can, for example, be a drain tube or a channel formed in the device. The conduit 222 allows fluid to drain away from the skin graft donor site and into the reservoir 216. Both the conduit and the reservoir can be detached once fluid drainage is sufficiently reduced. Optionally, the device further includes one or more filaments 218 between the port 106 and the reservoir 216, which can aid fluid management, e.g., drainage of fluid from the skin graft donor site 50 into the reservoir 216. Optionally, a vacuum source 220, e.g., a vacuum pump, can be coupled to the reservoir 216 to apply a suction force to the port 106 via the reservoir 216.

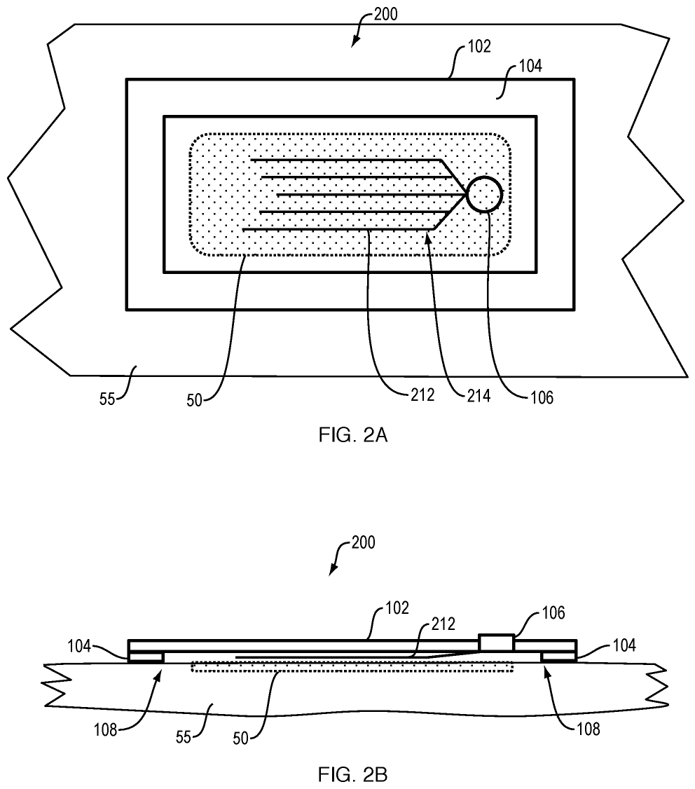

FIG. 3 illustrates a device 300 for treating a skin graft donor site, wherein the device has multiple ports 106a, 106b, 106c, 106d, 106e, 106f, 106g. The multiple ports are arranged so that the device has a higher density of ports (e.g., ports 106a-106f) in the sheet toward the boundary 108 than toward a center of the skin graft donor site (e.g., port 106g). As illustrated in the figure, the ports need not be of the same size, e.g., same diameter. In the example embodiment shown, ports 106a and 106b are circular and have the same diameter, which is larger than the diameters of circular ports 106c-106g. The higher density of ports 106a-106f toward the boundary at the adhesive 104 allows for fluid to drain from regions of the volume between the sheet and the skin graft donor site where fluid will tend to accumulate due to natural curvature of areas where skin graft donor sites are often made, such as a patient's leg.

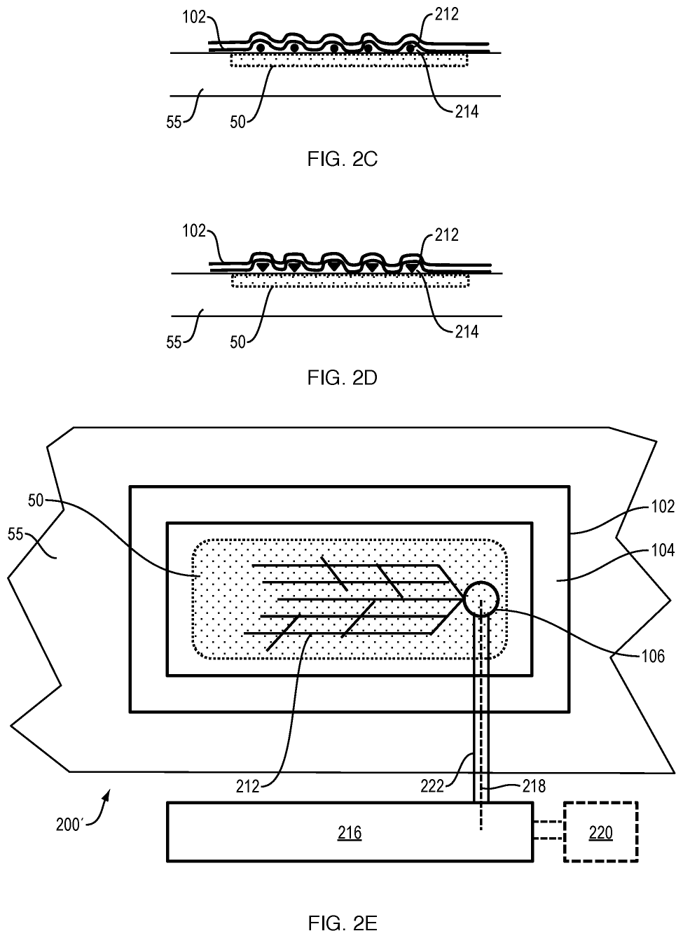

As described above with reference to FIG. 2E, the device can include a reservoir in fluid communication with the port. Various additional embodiments of devices including or being coupled to reservoirs are illustrated in FIGS. 4A, 4B, 5A, 5B, 6, 10A and 10B.

As illustrated in FIGS. 4A and 4B, a device 400 includes a bulb 424 defining a reservoir 416 and being configured to provide a suction force to apply a negative pressure through a port 106. The device 400 includes a sheet 102, an adhesive 104 and the port 106 integrated with the sheet, similar to other devices described herein, such as the device 100 of FIG. 1B. The bulb 424 can be removably coupled to the port 106 via a conduit 222. The bulb 424 can be operated manually (e.g., squeezed into a compressed state prior to coupling to the conduit 222) to remove fluid from the donor site 50 through the port 106 and into the reservoir 416 of the bulb. The device can further include a structure 426 to prevent clogging at the port 106. For example, a sieve-like structure or a membrane can be positioned at the port. FIG. 4B shows a patient having the device 400 applied to a skin graft donor site 50 on the patient's leg. Once drainage flow from donor site 50 is sufficiently reduced, the bulb 424 and conduit 222 can be disconnected from the port 106, leaving the remainder of the device in place to aid in the healing process of the donor site.

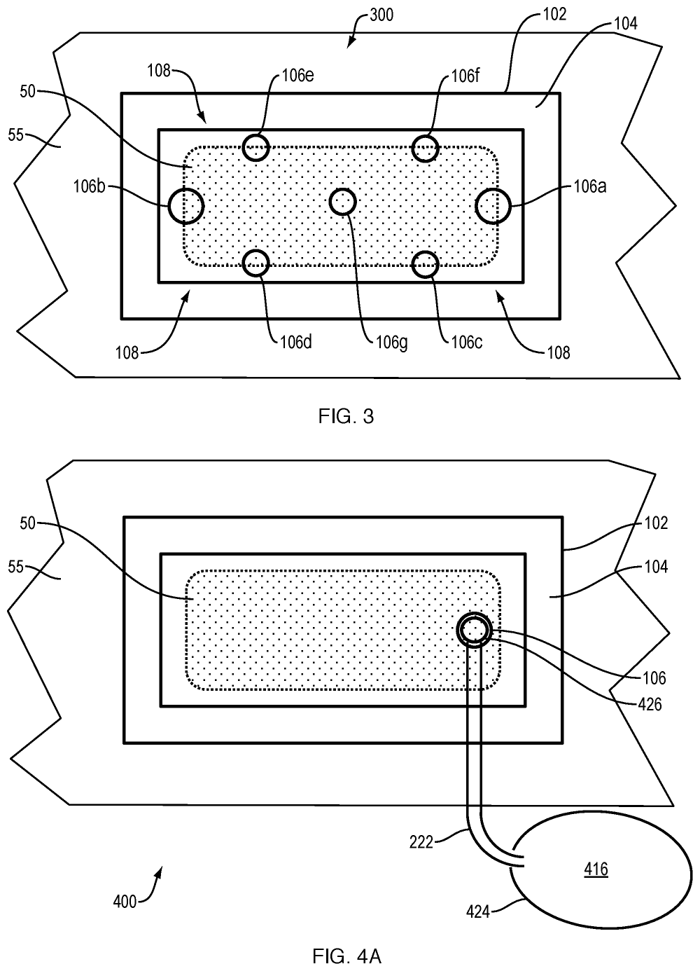

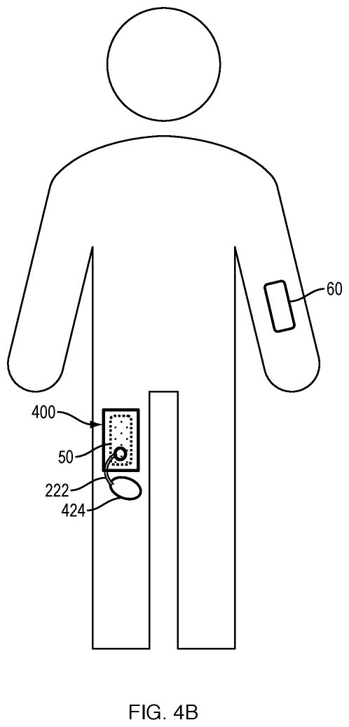

As illustrated in FIGS. 5A, 5B, and 6, embodiments of the device can include a reservoir that is positionable apart from the skin graft donor site.

FIGS. 5A and 5B illustrate a device 500 having a reservoir 516 in fluid communication with a port 106 for treating a skin graft donor site. The device 500 includes a sheet 102, an adhesive 104 and the port 106 integrated with the sheet, similar to the device of FIG. 1B. Fluid can drain from the port 106 via conduit 222 into the reservoir 516. Draining of fluid can be assisted by gravity, negative pressure, or combination of both. The device further includes a reservoir base sheet 528, configured to be fixedly coupled to skin apart from the skin 55 surrounding the skin graft donor site 50, and a reservoir top sheet 530 configured to be detachably coupled to the reservoir base sheet 528, e.g., via an adhesive 532 or other suitable coupling means. The reservoir 516 is defined between the reservoir base sheet 528 and the reservoir top sheet 530 in at least one location the reservoir top sheet is not coupled to the reservoir base sheet. By allowing the reservoir top sheet 530 to be detachable from the reservoir base sheet 528, the fluid aggregated into the reservoir 516 can be removed by detaching the reservoir top sheet 530, while not discomforting the patient by having to remove the reservoir base sheet 528. The reservoir base sheet 528 can be dried after the fluid is removed, and a fresh reservoir top sheet 530 can be applied.

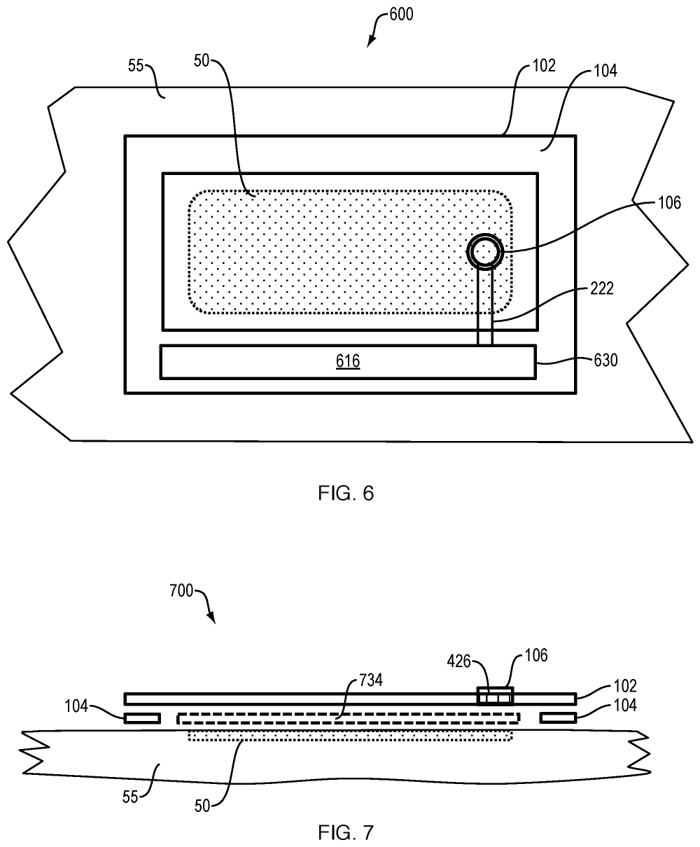

Any sheet of the device, such as the reservoir top sheet, can be formed from a vapor transmissible film, which can help in managing the fluid through evaporation between more active removal of fluid by a medical care practitioner or the patient, but also allows for gaseous exchange for purposes described below in reference to FIGS. 8A-8D.

As exemplified by device 600 illustrated in FIG. 6, a reservoir 616 can be integrated into the sheet. The device 600 includes a sheet 102, an adhesive 104 and a port 106 integrated with the sheet, similar to other devices described herein, such as the devices of FIGS. 1B, 2A, 3 and 4A. The reservoir 616 of the device can be defined between a reservoir base sheet and a reservoir top sheet, like the reservoir of the embodiment of FIGS. 5A and 5B. The sheet 102 covering the skin graft donor site can, for example, form one or the other of the reservoir base sheet and the reservoir top sheet. In the example shown, the sheet 102 forms the based sheet and a reservoir top sheet 630 is coupled to the sheet 102 to define the reservoir 616.

Embodiments of the device can further include filaments between the port and the reservoir, similar to the filaments illustrated in FIG. 2E, to aid is passage of fluid from the port to the reservoir.

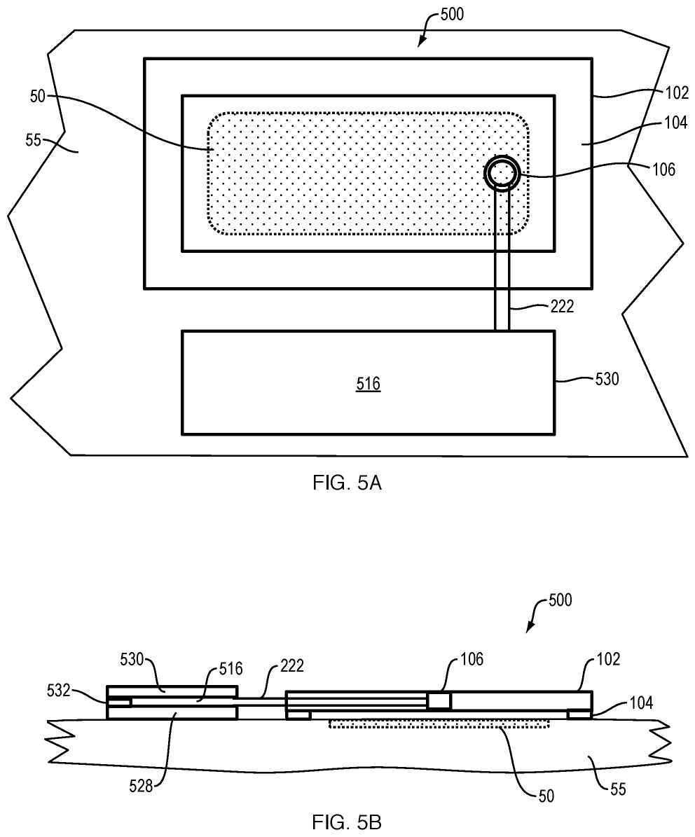

FIG. 7 is a schematic illustration of a device 700 for treating a skin graft donor site including additional material 734 positioned over the skin graft donor site and under the sheet of the device. In one example, the additional material includes bio-resorbable material configured to be positioned over the skin graft donor site 50 and under the sheet 102.

In some embodiments, the device can further include biomaterials with integrated growth factors to enhance epidermal healing coupled to the sheet 102.

The sheet 102 can include a drug or molecule embedded in the sheet for delivery to the skin graft donor site. A drug or a molecule can also be delivered to the skin graft donor site through the port 106.

In some embodiments, the device 700 can further include a structure 426 to prevent clogging at the port. For example, a sieve-like structure or a membrane can be positioned at the port. Preventing clogging can be particular useful when the device includes a biodegradable material or a hydrogel, which tend to break down and mix with the fluid from the donor site. The device can further include a removable drain device in fluid communication with the port to drain fluid from the skin graft donor site.

FIGS. 8A-8D schematically illustrate application of a device 800 to a skin graft donor site. The device 800 includes a sheet 102 and an adhesive 104. The adhesive 104 is provided on a backing sheet 836 and is configured to release from the backing sheet following contact of the adhesive to the skin surrounding the skin graft donor site 50. It should be understood that adhesive 104 that remains on the skin surrounding the skin graft donor site surrounds the skin graft donor site, not just on the left and right sides that is illustrate in FIGS. 8A-8D.

As illustrated in FIGS. 8A-8D, a method for treating a skin graft donor site include adhesively (104) coupling a sheet 102 to an area of skin 55 surrounding a skin graft donor site 50 to form a liquid impermeable boundary 108, and applying a suction force 110 through a port 106 integrated with the sheet 102 to produce a negative pressure between the sheet and skin graft donor site to manage fluid produced by the skin graft donor site.

In one embodiment, the invention includes a hybrid dressing device 900 that includes an occlusive PEG-diacrylate (PEGDA) dressing 902 with adhesive 904 around the edges. PEG is a cytocompatible material, non-adhesive, hydrophilic that allows gas and oxygen exchange, containing fluid exudate under the dressing in order to maintain moisture. PEGDA is a synthetic polymer that can be conveniently crosslinked upon exposure to ultraviolet (UV) light to generate flexible hydrogels. PEG-based hydrogels are highly tunable and biocompatible materials. PEGDA forms a soft and clear hydrogel, which is useful as a dressing material. It is a biologically inert and non-immunogenic substance. This hydrogel will have fluid drainage system in order to address the excess exudate from the donor site.

A previously published report has shown that a PEG and dl-lactic acid-co-glycolic acid (PLGA)-based hydrogel improved keratinocyte migration (Reference: Lee P Y, Cobain E, Huard J, Huang L., Mol Ther. 2007; 15(6):1189-1194). These hydrogels also supported the growth and proliferation of keratinocytes and improved re-epithelialization.

The hydrogel-based dressing physically provides a barrier against external factors that can potentially cause additional infection. Due to their high water content, hydrogel dressings can prevent water loss which is a critical aspect required for wound healing. Hydrogels are porous materials; therefore, they allow for adequate gaseous exchange over the wound. Hydrogels contain bioactive functional groups to enable cell adhesion and proliferation. A requirement of complete epithelialization of the wound is proliferation of dermal fibroblasts. These cells will proliferate on the proposed hydrogel.

The hydrogel dressing can i) be low-cost, ii) be flexible, iii) be easy to apply and remove, iv) be biocompatible, v) be sterilized easily, vi) be molded or cut into desired size and shapes, vii) possess tunable physical, chemical, and biological properties, viii) provide a moist environment for the wound without damaging the tissue, ix) allow for gaseous exchange, x) provide a physical barrier against external factors, xi) provide cushioning effect to the wound, xii) provide a cooling effect upon application which might reduce inflammation and pain, and xiii) have the potential to enhance epithelialization, angiogenesis, and vascularization.

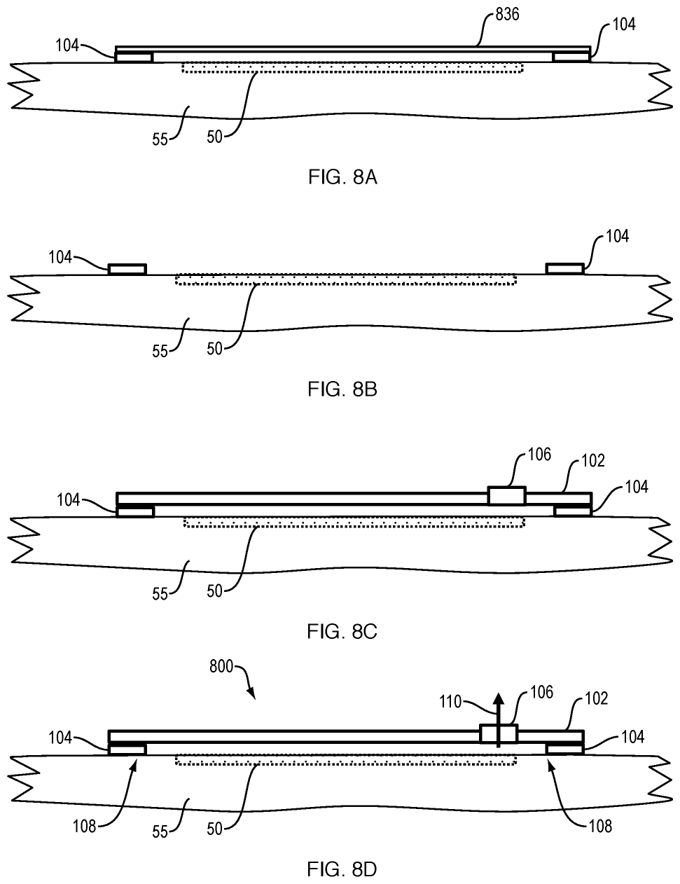

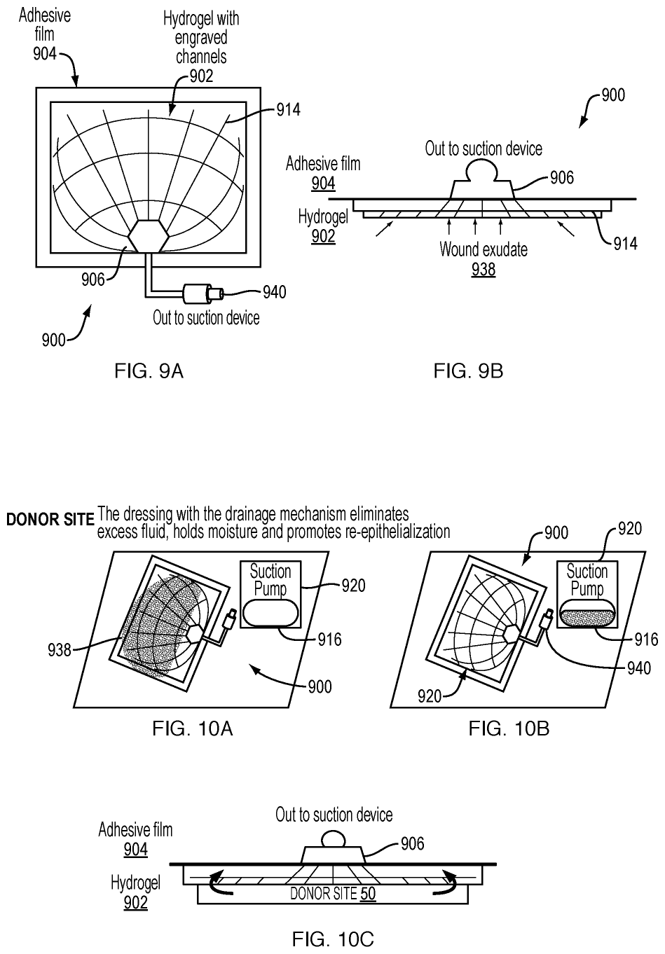

Moreover, the utility of the dressing is enhanced by a suction mechanism 920 (FIGS. 10A, 10B) that uses a channel system 914 engraved in the PEG sheet 902 (FIGS. 9A, 9B). The sub-millimetric channels 914 spread throughout the surface area of the wound allow contact with fluid exudate 938. Through capillarity and a suction force, excess fluid converges into one exit port 906. This port 906 can have a vacuum fitting 940 to connect to a manual or automated suction system, such as suction pump 920 with reservoir 916 (FIGS. 10A-10C).

The hybrid model keeps the interior environment intact, sterile free of excess fluid but moist. It requires minimal dressing changes and very low maintenance. It is lightweight and comfortable, which allows the patient to mobilize in the early post-operative period. The suction mechanism avoids the uncomfortable leakage around the dressing and the need for dressing exchange. This, in turn, minimizes nursing interventions, the risk of infection from manipulating the dressing, and patient pain.

In an embodiment that provides a transparent material as part of the sheet or of the entirety of the sheet, the transparent material allows medical practitioners and the patient to have visibility of and monitor closely both graft and donor site dressing. Due to the materials, the dressing is longer-lasting and inexpensive and meets all the principles listed above. Similarly, the moist environment of the volume between the sheet and skin graft donor site promotes keratinocyte migration.

In various embodiments, the invention also includes at least one of the following:
a) addition of growth factors to the hydrogel as well as trace elements for enhancing wound healing;
b) addition of any tissue engineered scaffold, natural or synthetic;
c) incorporation of a manual or electronic portable pump;
d) connection to either commercial suction pumps or hospital wall suction;
e) connection to manual mechanism such as syringes;
f) employment of sensor in the hydrogel for markers of apoptosis and necrotic tissue;
g) inclusion of dissolvable molecules of oxygen in the hydrogel; medications such as local anesthetics or promoters of wound healing
h) addition of Stem cells or mesenchymal cells embedded in the hydrogel; and
i) addition of cultured fibroblasts or keratinocytes in the hydrogel.

Other suitable applications of example embodiments of the invention include, for example, treatment of:

a) different types of fistulas (e.g., gastrointestinal, soft tissue, and urological);

b) highly exudative wounds, acute or chronic; and

c) burns.

In view of the foregoing descriptions of FIG. 9A through FIG. 10C, the following embodiments are now presented:

1. Embodiment 1 is a dressing for wound healing, comprising:
a) a hydrogel sheet, the hydrogel sheet defining channels and an opening, wherein the channels are in fluid communication with the opening;
b) a vacuum fitting in fluid communication with the opening, whereby the channels of the hydrogel sheet are in fluid communication with the vacuum fitting; and
c) an adherent at the hydrogel sheet, whereby the hydrogel sheet can be affixed to skin surrounding a wound, thereby causing the channels to be in fluid communication with the wound. In a specific embodiment, the channels may be in contact with the wound.

2. The dressing of embodiment 1, further including a vacuum source in fluid communication with the vacuum fitting.

3. The dressing of embodiment 1, further including at least one growth factor in the hydrogel sheet.

4. The dressing of embodiment 1, further including at least one wound-healing component in the hydrogel sheet such as cells, fragments of cells, growth factors, medications.

5. The dressing of embodiment 1, further including at least one natural or synthetic tissue-engineered scaffold at the hydrogel sheet.

6. The dressing of embodiment 1, further including means for connecting the vacuum fitting to a manual or portable suction pump, or a hospital wall suction port.

7. The dressing of embodiment 1, further including a fitting at the hydrogel sheet for injection of a fluid to the wound site such as medications, anesthetics or wound healing promoters.

8. Embodiment 8 is the dressing of embodiment 1, further including at the hydrogel sheet at least one sensor.

9. The dressing of embodiment 8, wherein the at least one sensor is selected from the group consisting of a marker for apoptosis, and a marker for necrotic tissue.

10. The dressing of embodiment 1, wherein the hydrogel sheet includes dissolved oxygen.

11. The dressing of embodiment 1, wherein the hydrogel sheet includes at least one of stem cells and mesenchymal cells.

12. The dressing of embodiment 1, wherein the hydrogel sheet includes at least one of cultured fibroblasts and keratinocytes.

13. Embodiment 13 is a method for treating a wound comprising the steps of:
a) adhering a hydrogel sheet to a wound site, the hydrogel sheet defining channels and an opening, wherein the channels are in fluid communication with the opening; and
b) applying intermittent vacuum through a fitting at the opening, whereby exudate is drawn from the wound, thereby treating the wound.

14. Embodiment 14 is the method of embodiment 13, wherein the wound is at least one member of the group consisting of: a skin graft recipient site; a skin graft donor site; a trauma site; a burn site; a surgical site; a fistula; an acute wound site; and a chronic wound site.

15. Embodiment 15 is the method of embodiment 14, wherein the wound is a fistula.

16. The method of embodiment 15, wherein the fistula is at at least one member of the group consisting of: gastrointestinal site; soft tissue site; and a urological site.

The relevant teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended s.

1. A device for treating a skin graft donor site, the device comprising:
a sheet having dimensions to cover the skin graft donor site and an area of skin surrounding the skin graft donor site;
an adhesive having properties of adhering to the skin and the sheet in a manner that forms a liquid impermeable boundary surrounding the skin graft donor site; and
a port integrated with the sheet that enables a suction force to be applied therethrough to produce a negative pressure between the sheet and skin graft donor site to manage fluid produced by the skin graft donor site.

2. The device of claim 1, further comprising filaments at the sheet and configured in the form of channels for fluid to pass therethrough from the skin graft donor site to the port.

3. The device of claim 2, wherein the filaments are embedded in the sheet.

4. The device of claim 1, wherein the port includes multiple ports.

5. The device of claim 4, wherein the multiple ports have a higher density in the sheet toward the boundary than toward a center of the skin graft donor site.

6. The device of claim 1, further comprising a reservoir in fluid communication with the port.

7. The device of claim 6, further comprising a bulb defining the reservoir and being configured to provide a negative pressure to apply the suction force through the port.

8. The device of claim 6, wherein the reservoir is integrated into the sheet and positionable apart from the skin graft donor site.

9. The device of claim 6, further comprising a reservoir base sheet, configured to be fixedly coupled to skin apart from the skin surrounding the skin graft donor site, and a reservoir top sheet configured to be detachably coupled to the reservoir base sheet, the reservoir being defined between the reservoir base sheet and the reservoir top sheet in at least one location the reservoir top sheet is not coupled to the reservoir base sheet.

10. The device of claim 9, wherein the reservoir top sheet is formed from a vapor transmissible film.

11. The device of claim 6, further comprising filaments between the port and the reservoir.

12. The device of claim 1, further comprising bio-resorbable material configured to be positioned over the skin graft donor site and under the sheet.

13. The device of claim 1, further comprising a structure to prevent clogging at the port.

14. The device of claim 1, wherein the sheet includes a material selected from a group of materials consisting of: a polyurethane, an organic polymer, a hydrogel.

15. The device of claim 1, further comprising biomaterials with integrated growth factors to enhance epidermal healing coupled to the sheet.

16. The device of claim 1, wherein the port is sized to pass up to 100 ml of fluid per day.

17. The device of claim 1, wherein the negative pressure is less than 100 mm Hg.

18. The device of claim 1, wherein the adhesive is on the sheet other than over a portion of the sheet expected to cover the skin graft donor site.

19. The device of claim 1, wherein the adhesive is provided on a backing sheet and is configured to release from the backing sheet following contact of the adhesive to the skin surrounding the skin graft donor site.

20. The device of claim 1, wherein the sheet includes transparent material to allow visualization of the donor site without removal of the sheet.

21. The device of claim 1, wherein the sheet includes a drug or molecule embedded in the sheet for delivery to the skin graft donor site.

22. The device of claim 1, further comprising a removable drain device in fluid communication with the port to drain fluid from the skin graft donor site.

23. A method for treating a skin graft donor site, the method comprising:
adhesively coupling a sheet to an area of skin surrounding a skin graft donor site to form a liquid impermeable boundary; and
applying a suction force through a port integrated with the sheet to produce a negative pressure between the sheet and skin graft donor site to manage fluid produced by the skin graft donor site.

24. The method of claim 23, further comprising delivering a drug or molecule to the skin graft donor site, wherein the drug or molecule is embedded in the sheet or delivered through the port.
